(19) United States
(12) Patent Application Publication
(10) Pub. No.: US 2014/0113306 A1
(43) Pub. Date: Apr. 24, 2014

(54) DIAGNOSTIC AND SCREENING METHODS FOR INFLAMMATION

(71) Applicant: Rutgers, The State University of New Jersey, New Brunswick, NJ (US)

(72) Inventors: Beatrice Haimovich, North Brunswick, NJ (US); Zhioyng Zhang, Edison, NJ (US); Louis Amorosa, Somerville, NJ (US)

(73) Assignee: Rutgers, The State University of New Jersey, New Brunswick, NJ (US)

(21) Appl. No.: 14/056,642

(22) Filed: Oct. 17, 2013

Related U.S. Application Data
(60) Provisional application No. 61/714,922, filed on Oct. 17, 2012.

(51) Int. Cl. G01N 33/569

(52) U.S. Cl. CPC G01N 33/56972
USPC 435/7.1

(57) ABSTRACT

This invention is directed to methods, kits and compositions for the diagnosis and treatment of conditions associated with sub-clinical inflammatory conditions such as diabetes mellitus.

### RELATED APPLICATIONS

This application claims priority under 35 U.S.C. §119(e) from Provisional U.S. patent application Ser. No. 61/714,922 filed on Oct. 17, 2012. The entire contents of each of which are herein incorporated, by reference.

### GOVERNMENT SUPPORT

This invention was made with government support under grant number NIEHS ES005022 awarded by Environmental and Occupational Health Sciences Institute (EOHSI). The government has certain rights in the invention.

### FIELD OF THE INVENTION

This invention relates to methods, kits and compositions for the diagnosis and treatment of inflammatory conditions and related diseases such as diabetes. More specifically, the invention relates to using blood cells biomarkers for early diagnosis and treatment of chronic inflammatory conditions associated with diseases such as diabetes.

### BACKGROUND OF THE INVENTION

Diabetes is a risk factor for morbidities that include heart attack, stroke, and kidney failure and as such, a cause for an ever increasing economic burden on society. Disrupted glucose homeostasis and insulin signaling are characteristic of both type 1 and type 2 diabetes mellitus. However the link between chronic low-grade inflammation and circulating immune cell activation in patients with diabetes is undetermined.

Recent studies conducted in a Toll-like receptor (TLR4)-deficient mouse model of high-fat diet (HFD)-induced insulin resistance has suggested that TLR4 signaling may contribute to the pathogenesis of type 2 diabetes and that weight-gain in the HFD mouse model may be associated with an increase in circulating endotoxin levels. Chronic administration of a low concentration of endotoxin to mice for 4 weeks has been shown to trigger weight-gain and insulin-resistance. Yet, the state of art is not clear whether modest increases in circulating endotoxin levels observed in type 1 and type 2 diabetic patients contribute to immune cell activation.

Despite advances in our understanding of diabetes and management of the disease, there are no systemic inflammatory indicator(s) or diagnostic tools that can reliably predict and identify which subjects are at risk of developing the disease. Given the alarming projected increase in individuals at risk, there is an urgent need to identify novel, minimally-invasive biomarkers for subclinical inflammatory responses that are predictive of diabetes. The present invention addresses such need.

### SUMMARY OF THE INVENTION

This invention provides methods, kits and compositions for the diagnosis and treatment of conditions associated with sub-clinical inflammatory conditions. The invention is also directed to methods, kits and compositions for diagnosis and treatment of such disease as diabetes mellitus. More specifically, a number of biomarkers including cells, proteins, cytokines, and fragments thereof are identified in subjects at risk of developing diabetic or pre-diabetic conditions that can be employed as a predictor for the subjects at risk of developing diabetes.

In at least one aspect of the invention, peripheral blood cells and their respective cellular proteins serve as a biomarker for diagnosis of inflammatory conditions associated with diabetes. In at least one embodiment, these peripheral blood cells are peripheral blood leukocytes (PBLs). In another embodiment, the biomarkers include cellular proteins and transcription factors such as Hypoxia-inducible factor (HIF), including subunit HIF-Iα, Protein Kinases such as Akt (Akt), Ribosomal p70 S6 kinase 1 (S6K1); insulin receptor substrate (IRS) including IRS-1, their phosphorylated and unphosphorylated forms, or any other post-translational modifications thereof.

In another aspect of the inventions kits and products are described that can be used for early diagnosis of diabetes or subject's predisposition to diabetic conditions. In a preferred embodiment, the present invention is directed to in vitro assays that can be used to induce biochemical outcomes similar to those observed in leukocytes obtained from patients with an acute inflammatory state or chronic inflammatory disease such as diabetes. In yet another embodiment, methods of screening natural or synthetic compounds that can reverse or induce outcomes related to an acute inflammatory state or chronic inflammatory disease such as diabetes are described.

In another aspect of the invention, methods of profiling the expression and/or activity of specific cellular proteins in peripheral blood leukocytes, or leukocyte subpopulations are described. In at least one aspect of the invention, health care providers are able to determine patient state of health and further monitoring treatment efficacy and outcomes. In at least one embodiment, methods for profiling, screening and diagnosing a subject suffering from subclinical inflammatory condition are assessed by (a) providing a biological sample from a subject, (b) measuring the level of a leukocyte biomarker in the sample, (c) comparing the measured level with a baseline or normalized level of the same biomarker from a control subject(s) that is not suffering from the inflammatory condition, wherein a deviation of the level from the baseline and/or normalized is an indication of subclinical inflammatory condition. In one embodiment, the subclinical inflammatory condition is related to and/or induced by diabetes mellitus. In another embodiment, the baseline or normalized measurements of biomarkers are population based.

In another aspect of the invention, a group of peripheral blood leukocytes cellular proteins are identified to serve as biomarkers for diagnosis of an acute inflammatory state, sub-clinical (low-grade) inflammation (such as a change in body temperature ≦1 C°, or a change in heart rate ≦30 bpm), chronic inflammation or inflammatory diseases such as diabetes before and after the appearance of the defined disease biomarkers. In another embodiment, the biomarker include but are not limited to ATP, AMPKα, HIF-1α, Glut3, S6K1, TLR4, Akt, caspase-1, IRS-1, Raptor, and MMP9, their phosphorylated and non-phosphorylated forms, or any other post-translational modifications thereof.

In another aspect of the invention, peripheral blood leukocyte proteins are identified for determining the clinical trajectory of patients experiencing sub-clinical or low-grade inflammation for developing a defined disease that is associated with inflammation. In a more preferred aspect of the invention, the disease associated with the inflammation is diabetes, arthritis, or cardiovascular disease. In an embodiment, peripheral blood biomarker levels are used for monitoring treatment efficacy and outcomes of any such disease. In a more preferred embodiment, the peripheral blood biomarkers are leukocyte cellular proteins.

In yet another aspect of the present invention, methods of diagnosing and objectively evaluating diseases, the progression of such diseases and namely inflammatory diseases are described herein. In at least one embodiment, the invention is directed to methods of diagnosing and evaluating the diseases before and/or after, the appearance of biochemical markers described herein of the disease. In at least another embodiment, the invention provides for methods of objectively evaluating predisposition to an inflammatory disease.

In at least another embodiment, positive response to therapy is expected to treat, prophylactically treat, prevent, alter and/or reverse the expression profile of the biomarkers and the diseases associated with such biomarkers. The presently disclosed biomarkers can be evaluated by a variety of means known in the art to determine a specific cellular protein subset, including but not limited to Western blot analyses, ELISA, flow cytometry and multiplex assays. In at least one embodiment, the biomarker is a protein kinase.

In yet another aspect of the invention, novel methods are described for profiling, screening and diagnosing a subject risk of developing diabetes by (a) providing a biological sample from a subject, (b) measuring the levels of at least one leukocyte biomarker in the sample using a plurality of assays, and (c) comparing the measured levels in each of said assays with a baseline or normalized level of a control subject that is not suffering from diabetes. In at least one embodiment, the profiling, screening and diagnosis is determined based on subject's presence or absence of a biomarker or change or deviation in biomarker's levels from the baseline or normalized level. In at least one embodiment, the biomarker deviation includes degradation, enhanced expression, decreased expression, phosphorylation or dephosphorylation, or any other post-translational modification of said biomarkers from subject's baseline or normalized level of the same. In at least one embodiment, the biomarker deviation includes changes in cellular ATP levels, degradation of AMPKα, expression of HIF-1α, activation of caspase-1, enhanced expression of TLR4 and MMP9, enhanced phosphorylation of Akt, enhanced phosphorylation of S6K1, dephosphorylation of Raptor, and enhanced phosphorylation of IRS-1 on serine residues.

In at least another aspect of the invention, inventors describe methods for screening for a therapeutic agent useful in treatment or prophylactic treatment of an inflammatory disease by (a) providing a number of candidate compounds; (b) providing a pool of leukocytes, (c) establishing the baseline expression of any one of the proteins selected from the group consisting of AMPKα, HIF-1α, Glut3, S6K1, TLR4, Akt, caspase-1, IRS-1, Raptor, and MMP9, their phosphorylated and unphosphorylated forms, or any other post-translational modifications thereof, in said leukocytes, (d) contacting each candidate compound with the pool of leukocytes; (e) determining the expression level of any one of said protein, and (f) selecting, the compound that causes a deviation of the level from the baseline expression of said protein as a candidate for the therapeutic agent. A more detailed description of the invention is provided herein below.

### BRIEF DESCRIPTION OF THE DRAWINGS

FIG. 1. provides for In vivo endotoxin-induced responses in humans. Subjects (n=5 per group) were challenged with the indicated endotoxin dose or saline (placebo group). Each subject was assigned a specific color/symbol code that can be followed throughout FIGS. 1 and 2. (A) Endotoxin induced dose-dependent systemic and cytokine responses. The panels in the left hand column represent mean responses. (B-D) Temporal change in leukocyte transcripts were examined for a subset of the subjects described in A. (B) Gene expression analyses identified ˜175 common genes that were either induced or suppressed at least 2-fold in leukocytes from subjects (n=3) challenged with endotoxin at 1 ng/kg. The changes in gene expression peaked between 2 and 6 hours. (C) RPL/RPS gene expression patterns observed in leukocytes challenged with an in vivo endotoxin dose of 1 ng/kg (lanes C, D and F) or 0.1 ng/kg (lanes b, c, d and e). Green and red indicate, respectively, a decline or an increase in gene expression relative to time 0. (D) Shown are expression patterns of select genes, which are highlighted in panel B.

FIG. 2. indicates that endotoxin causes metabolic dysfunction in human leukocytes. Blood samples were obtained at the times indicated, from subjects described in FIG. 1 who were challenged with endotoxin at either (A) 1 ng/kg or (B) 0.1 ng/kg. Leukocyte lysates containing equal protein amounts were subjected to Western blotting or ATP analyses. (C) Schematic contrasting the in vivo endotoxin response at doses of 0.1 ng/kg or 1 ng/kg. Both low and high dose endotoxin initiate metabolic dysfunction in human leukocytes. In contrast, only high endotoxin dose triggers robust inflammatory responses in all subjects.

FIG. 3. indicates that ex vivo endotoxin induced responses in human leukocytes. (A) Blood samples were treated with endotoxin (LPS; 10 ng/kg) for the indicated time. (B-D) Blood samples were untreated (control), treated for 2 hours with DMSO (vehicle), or the specified inhibitors prior to the addition of endotoxin (10 ng/ml). The inhibitors tested included: LY294002 (LY), Rapamycin (RAPA), YC-1, acriflavine (ACF), metformin (Met) and SRT1720 (SRT). Leukocytes were isolated at the indicated time post-endotoxin treatment and lysed. Lysates were subjected to Western blotting analyses, and where shown, ATP analyses. Details are provided in the supplement. (E) Proposed model for endotoxin-induced metabolic derangement in human leukocytes.

FIG. 4. shows that PBL obtained from a subset of diabetic patients exhibit a metabolic dysfunction signature (MDS) that includes AMPKα degradation, HIF-1α expression and autophagy, revealed by changes in LC3-I/II expression. In addition, PBL from the same subset of subjects exhibit caspase-1 activation (cleavage), enhanced TLR-4 and MMP9 expression. Blood samples were obtained from non-diabetic (N), type 1 (1) and type 2 (2) diabetic patients seen at Rutgers RWJMS clinics. Patient demographics are shown in Table 2. The researchers were blinded to the clinical characteristics of the study participants while the samples were being analyzed. (*) denotes a non-diabetic control (Cntrl) donor used multiple times. Arrowhead denotes a non-diabetic patient with a HbAlC of 6% and fasting glucose of 117 mg/dL. Leukocytes were isolated, lysed, and analyzed by immunoblotting. Immunoblotting for actin was used to confirm equal protein loading. Increases in LC3-II, HIF-1α, TLR4 and MMP9 expression, as well as AMPKα cleavage and caspase-1 cleavage/activation were all detected in 7 of 13 type 2 diabetic patients and 3 of 3 type 1 diabetic patients.

FIG. 5, provides that the leukocytes from type 1 and type 2 diabetic patients exhibit changes in phosphorylation state of Akt, S6K1, Raptor, and IRS-1. Blood samples were obtained from non-diabetic (N), type 1 (1) and type 2 (2) diabetic patients seen at Rutgers RWJMS clinics. Patient demographics are shown in Table 2. The researchers were blinded to the clinical characteristics of the study participants while the samples were being analyzed. (*) denotes a non-diabetic control (Cntrl) donor used multiple times. Arrowhead denotes a non-diabetic patient with a HbAlC of 6% and fasting glucose of 117 mg/dL. Leukocytes were isolated, lysed, and analyzed by immunoblotting for the levels and phosphorylation state of the specified proteins. Immunoblotting for actin was used to confirm equal protein loading.

FIG. 6. shows that diabetic patients' plasma contain a soluble and transferable leukocyte activator component. (A) Blood samples from a non-diabetic (N) and a type 2 diabetic patient were separated into plasma and cellular fractions (cellular F.) and mutually exchanged. Thus, plasma derived from the non-diabetic donor was added to the diabetic patient cellular fraction, and vice versa. Leukocytes were isolated at indicated time post-exchange, lysed and analyzed by immunoblotting. (B) Blood samples from a non-diabetic donor were treated with insulin (0.1 unit/ml) for the indicated time (lanes 1-7) or with endotoxin for 2 hours (lane 8) (C) Blood samples from a non-diabetic donor were untreated (UN) or treated for 1 hour with polymyxin (50 μg/ml) or CL-095 (30 μM). Endotoxin (Endo; 10 ng/ml) was next added to specified samples (lanes 2-4). Leukocytes were isolated 2 hours tater. (D and E) Blood samples obtained from a non-diabetic donor and two type 2 diabetic patients (identified as diabetic 1 and diabetic 2) were all separated into plasma and cellular fraction. The cellular fractions were either untreated (lane 4) or treated with polymyxin (lanes 5 and 8) or CLI-095 (lanes 6 and 9) for 1 hour. The samples shown in lanes 1-3 were not subjected to plasma exchange. (D) Plasma derived from diabetic 1 (lanes 4-6) or diabetic 2 (lanes 7-9) was added to the non-diabetic donor cellular fractions and (E) vice versa. The leukocytes were isolated at 4 hours post-exchange. Samples were analyzed as in (A).

FIG. 7. provides the Toll-like receptor 4 signaling pathway in human leukocytes and the antagonistic effect of insulin. (A) Blood samples were treated with endotoxin (10 ng/ml) for the indicated time. (B) Blood samples were untreated (UN), or treated with DMSO (vehicle; 0.05%; 2 hours), LY294002 (LY; 10 μM; 30 min), rapamycin (RAPA; 100 nM, 2 ‘hours), YC-1 (30 μM; 30 min), or acriflavine (ACF; 5 μM; 2 hours). The samples shown in lanes 3-8 were subsequently treated with endotoxin (10 ng/ml) for 2 hours. (C) Blood samples were untreated (UN; lane 1), treated with endotoxin (10 ng/ml; lane 2) for 2 hours, or with insulin (1 unit/ml) for 2 hours (lane 3) or 1 hour (lane 4). The samples shown in lanes 5-9 were treated with endotoxin (10 ng/ml) plus insulin at the indicated concentration (1-0.05 unit/ml) for 2 hours, (D) Blood samples were treated with insulin (0.1 unit/ml) plus endotoxin (10ng/ml) for the indicated time, or with endotoxin alone (Endo, 10 ng/ml) for 2 hours. Leukocytes were isolated, lysed, and analyzed as described in FIG. 5. (E) Model describing the pattern of signaling indicators detected in leukocytes treated with endotoxin as compared to insulin. Leukocytes treated with endotoxin plus insulin at a concentration ≧0.25 unit/ml exhibited a signaling pattern similar to that seen in blood leukocytes treated with insulin alone, while those co-treated with insulin at a concentration <0.1 unit/ml exhibited the pattern observed in leukocytes treated with endotoxin alone.

### DETAILED DESCRIPTION OF THE INVENTION

“Increased expression” or “enhanced expression” refers to increasing (i.e., to a detectable extent) concentration/amount/level of the polypeptide or protein encoded by a specific gene. As used herein “upregulated,” and “upregulation,” refer to increased expression of a gene and/or its encoded polypeptide or protein. Conversely, “downregulation,” or “decreased expression” as used herein, refers to decreased expression of a gene and/or its encoded polypeptide.

The upregulation or downregulation of gene expression can be directly determined by detecting an increase or decrease, respectively, in the level of mRNA for the gene, or the level of protein expression of the gene-encoded polypeptide, using any suitable means known to the art as compared to controls.

The term “expression,” as used herein, refers to nucleic acid and/or polypeptide expression.

The term “leukocytes” or white blood cells refer to cells of the immune system that are involved in defending the body against both infections and foreign materials. They exist throughout the body, including the blood, the lymphatic system, and within organs.

The term “subject” is a mammal, including human and a non-human mammal.

HIF-1 is a heterodimeric transcription factor composed of an inducible a subunit (HIF-1α), and a constitutively expressed HIF-1β subunit. Endotoxin/TLR-4 induce HIF-1α expression and activate HIF-1 in leukocytes under either hypoxic or normoxic conditions. Though HIF-1 activation contributes to cell survival when total oxygen availability is limited, sustained HIF-1 activation has deleterious outcomes. HIF-1 regulates the transcription of numerous genes, including a subset that controls metabolism by suppressing mitochondrial function and enhancing glycolysis. HIF-1 can also activate autophagy, a process that enables digestion of cellular macromolecules and organelles through specialized vacuoles known as autophagosomes. During periods of nutrient deficiency, autophagy products are used by mitochondria to produce ATP. The microtubule associated protein light chain-3 (LC3-I) is a cytosolic protein that participates in autophagosomes formation. The increase in expression of the modified form of known as LC3-I, is indicative of autophagy.

AMPK is a serine-threonine kinase, composed of AMPK-α, -β, -γ subunits, that is activated when the cellular ATP levels are low. AMPK regulates numerous targets, including PGC-1, a transcription factor that induces mitochondrial biogenesis. AMPK can also regulate autophagy. The present inventors were the first to show that endotoxin induces AMPK (degradation in liver of mice. The molecular mechanism linking TLR-4 to AMPK (degradation is currently undetermined. MMP9 is matrix metalloprotease-9.

In general, human homologs and alleles typically will share at least 80% nucleotide identity and/or at least 85% amino acid identity to the characterized rat sequences of the invention. In further instances, human homologs typically will share at least 90%, 95%, 98% or even 99% amino acid identity to the characterized sequences. The homology can be calculated using various, publicly known methodology or software tools.

Screening methodologies for human related protein, biomarkers, genes, or cells described herein, may be performed using stringent conditions, together with a probe. The term “stringent conditions” as used herein refers to parameters with which the art is familiar. There are other conditions, reagents, and so forth which can be used, and would result in similar degree of stringency. The skilled artisan will be familiar with such conditions, and thus they are not given here. The skilled artisan also is familiar with the methodology for screening cells and libraries for expression of such molecules which then are routinely isolated, followed by isolation of the pertinent nucleic acid molecule and sequencing.

The present invention involves the discovery of the correlation between the leukocytes cellular proteins from diabetic patients and the activated phenotype that is indicative of TLR4 activation and sensitive to insulin availability, in view of this invention, it is believed that certain biomarkers can be used to diagnose and or develop molecular screening methods for treatment diabetes. In addition, the present invention describes methods for using these biomarkers or homologs thereof in the diagnosis of any of the foregoing diabetic conditions

Chronic, low-grade inflammation has been associated with the pathogenesis of diverse human diseases, including diabetes; however, there are currently few in vitro models that can reproduce the biochemical changes induced in response to low-grade inflammation in vivo. Leukocytes, which include neutrophils and monocytes, are key mediators of host-inflammatory responses. The possibility that circulating leukocytes have an activated phenotype in diabetic patients has not been explored. Endotoxin (lipopolysaccharide; LPS) is a ligand of Toll-like receptor 4 (TLR-4). Endotoxemia is an experimental model of acute systemic inflammatory responses in human and model organisms such as mice.

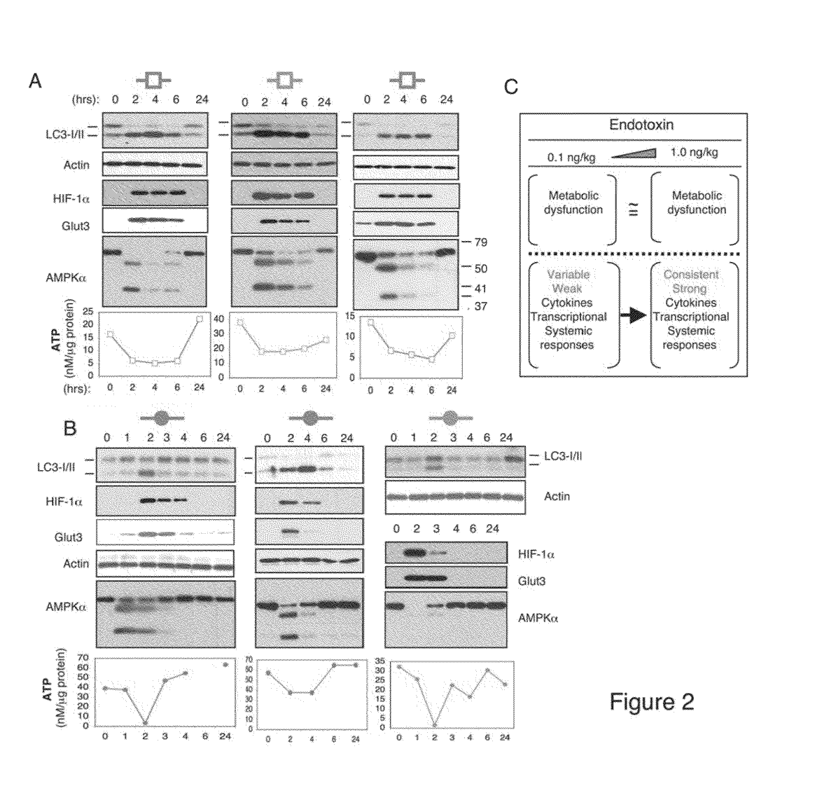

In the human model, subjects are challenged with a bolus endotoxin dose of up to 4 ng/kg and responses are monitored over a period of up to 24 hours post-challenge. For the first time in the art, the present inventors describe that endotoxin triggers an increase in autophagy. HIF-1α and Glut3 expression, meanwhile, causing a decline in AMPK α expression in human peripheral blood leukocytes (FIG. 2A).

Recent data have suggested an association between diabetes and chronic low-level endotoxemia in human subjects, providing a potential link between sub-clinical inflammation and metabolic diseases. In experimental endotoxemia, healthy subjects are challenged with a bolus dose of purified Escherichia coli endotoxin (lipopolysaccharide; LPS). Endotoxin induced responses are dose-dependent. However, the effects of low-endotoxin levels that are below the threshold associated with systemic responses are currently undetermined.

In one aspect of the present invention, the inventors characterize cellular responses that are triggered in human subjects in vivo following an endotoxin dose at which the well-described inflammatory signatures are not detected. Accordingly, in one embodiment, the inventors identified biomarkers that can detect sub-clinical inflammatory responses.

At least one aspect of the present inventions are directed to method for screening and diagnosing a subject suffering subclinical inflammatory response by (a) providing a biological sample from a patient, (b) measuring the sample for expression of a leukocyte biomarker or a homolog thereof, to comparing the measured level with a baseline or normalized level of the biomarker obtained from a patient that is not suffering from the inflammatory condition, wherein the existence or the modification of such biomarker from the baseline is an indication of preclinical inflammatory disease.

Exemplary inflammation-related disease includes endocrine diseases such as diabetes mellitus, disorders of adrenal glands, and pituitary as well as gonadal glands. Other examples of inflammatory related disease include infections such bacterial-induced inflammation including Chlamydia-induced inflammation, viral induced inflammation; cardiovascular disorders such as vascular diseases, coronary artery disease, aneurysm, vascular rejection, arterioselerosis, atherosclerosis including cardiac transplant atherosclerosis, myocardial infarction, embolism, stroke, thrombosis including venous thrombosis, angina including unstable angina, coronary plaque inflammation, angioplasty, stent placement, and the like. Exemplary angiogenesis-related disorders include neoplasia including metastasis, benign and malignant tumors, and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, liver cancer, bladder cancer, pancreatic cancer, ovarian cancer, prostate cancer, cervical cancer, lung cancer, breast cancer and skin cancer. In at least one aspect of the present invention, the leukocyte biomarkers maybe employed to identify sub-clinical inflammatory responses that are associated with such chronic disease that effect gastroenterology, respiratory, hematology and neurology systems. In a more preferred embodiment, the sub-clinical inflammatory response is associated with an endocrine disease, preferably diabetes mellitus, namely diabetes type 1 and type 2.

In yet another aspect of the invention, the invention is directed to methods of assessing biological samples that are obtained from lymphatic system or bone marrow. More particularly, these samples are screened for existence of biomarkers including but not limited to leukocyte's ATP, AMPKα, HIF-1α, Glut3, TLR4, caspase-1, and MMP9, and homologs thereof. In a preferred embodiment, the biomarkers are ATP or leukocyte protein such as, AMPKα, HIF-1α. Glut3, S6K1, TLR4. Akt, caspase-1, IRS-1, Raptor, and MMP9 their phosphorylated and unphosphorylated forms, or any other post-translational modifications thereof,

In yet another embodiment, the screening methodology is designed to detect modifications of the levels of biomarker against a control, baseline or population normal levels. In a preferred embodiment, the screening methodology is designed to detect such modification as elevation of ATP, degradation of AMPKα, expression of activation of caspase-1, enhanced expression of TLR4 and MMP9 or any combinations thereof.

In another aspect of the present invention, a method for screening and diagnosing a subject risk of developing diabetes is contemplated by (a) providing a biological sample from a patient, (b) measuring, the sample for expression of at least one leukocyte biomarker in a plurality of assays, and (c) comparing the measured levels in each of said assays with a baseline or normalized level of a subject that is not suffering from the diabetes. In this aspect of the invention, elevation of ATP, degradation of AMPKα, expression of HIF-1α, activation of caspase-1, enhanced expression of TLR4 and MMP9, from the baseline or normalized level detected. Any such observation is indicative of the subject's risk of developing diabetes.

In yet another embodiment, the baseline or normalized level of subjects that are not suffering from the diabetes is a measurement of HbAlC and fasting glucose levels. In a more preferred embodiment, the baseline HbAlC is below 6.5 and the baseline fasting plasma glucose is less than 126 mg/dl. In a more preferred embodiment, the method is directed towards subjects at risk of developing type II diabetes. In such embodiment, type II patients will show an increase in TLR-4 expression.

In yet another aspect of the present invention, a method for screening for a therapeutic agent useful in treatment of an inflammatory related disease by (a) providing a number of candidate compounds; (b) providing a pool of leukocytes, (c) establishing the baseline expression of any one of the proteins selected from the group consisting of AMPKα, HIF-1α, Glut3, TLR4, caspase-1, and MM P9 in said leukocytes, (d) interacting each candidate compound with the pool of leukocytes; (e) determining the modification or deviation from the baseline expression of any one of said protein or any homologs thereof, and (f) selecting the drug that provides modification of the baseline expression of said proteins. In a more preferred embodiment, this method is designed to identify the therapeutic agents that are useful for treatment of endocrinology disease, namely diabetes mellitus. In this embodiment, the deviation from the baseline expression is selected from the group consisting of reversing degradation of AMPKα, reversing expression of reversing activation of caspase-1, reversing expression of TLR4 and MMP9 and any combinations thereof.

Another aspect of the invention pertains to novel agents identified by the above-described screening assays and uses thereof for treatment as describe herein. For example, any active compound in the form of nucleic acid molecule, polypeptide, antibody, small molecule that is identified by the presently described assay, can be incorporated into a pharmaceutical composition suitable for administration. Such composition typically contains a pharmaceutically acceptable carrier which as used herein is intended to include solvents, dispersion media, coating, and antibacterial, anti-fungal, isotonic, pH adjuster, or a suitable delaying agents. In at least one embodiment, inhibitors of low-grade inflammation can include agents that suppress HIF-1α, Glut3, and/or MMP9 expression, and prevent autophagy. Such inhibitors can be produced using methods which are generally known in the art, in particular, methods known in the art to produce antibodies or to screen libraries of pharmaceutical agents to identify drug candidates which can reverse the endotoxin inflammatory response in diabetic patients. In at least one aspect of the invention, the inventors show that the decline in AMPK expression of patients at risk of developing diabetes is clue to its degradation (FIG. 2A). These findings are particularly significant since HIF-1α and AMPK are central regulators of cellular bioenergetics.

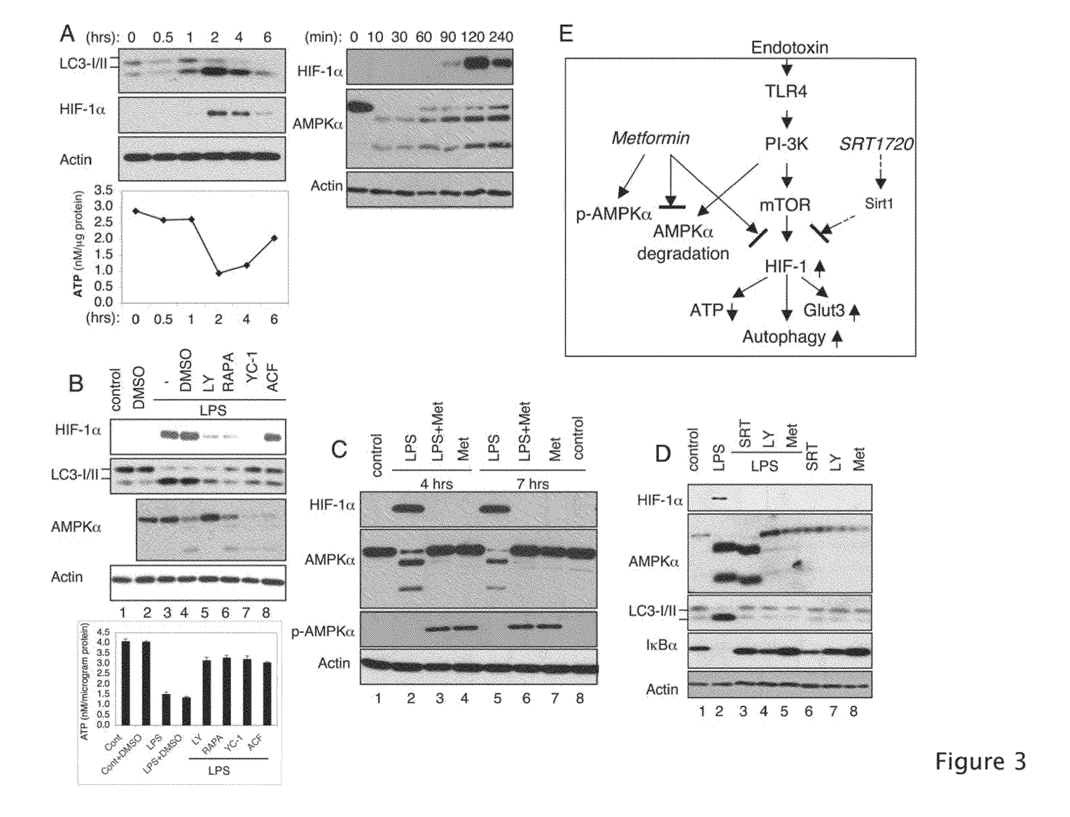

In at least another embodiment, the inventors describe methods for treating human leukocytes with endotoxin (5-10 ng/ml) ex-vivo/in vitro. The ex-vivo/in vitro endotoxin-treated leukocytes exhibit biochemical changes in HIF-1α, Glut3, and AMPKα expression, autophagy, and ATP levels, which reproduce the changes observed in leukocytes exposed to endotoxin in vivo (FIG. 3A).

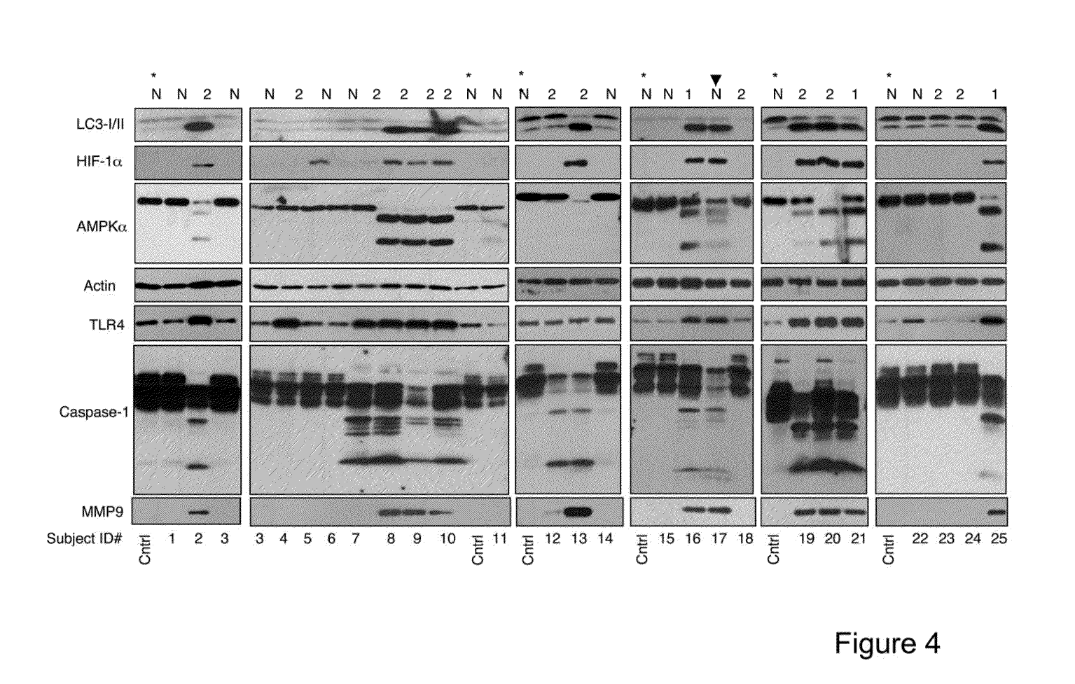

In another embodiment, methods of reproducing the protein expression pattern seen in vivo and in vitro in leukocytes exposed to endotoxin was reproduced in leukocytes from patients with diabetes (FIG. 4). In another embodiment, such expression system can be used as an assay for identifying candidate therapeutic agents that can suppress the endotoxin induced low-grade or subclinical inflammatory response.

The present inventors have discovered that AMPK, HIF-1, and autophagy are all involved in metabolic processes, to that extent, the aberrant expression pattern of this group of proteins in human PBL are referred herein as the metabolic dysfunction signature (MDS). At least in one embodiment, MDS (i.e., AMPK α degradation, HIF-1α expression and autophagy) in PBL from healthy subjects challenged with in vivo endotoxin doses above or below the threshold were needed to trigger systemic inflammatory responses. In another aspect of the present invention, MDS in PBL from a subset of subjects at high risk for developing diabetes and diabetes patients were detected, but not in PBL from healthy control subjects or non-diabetic patients.

In yet another aspect of the invention, inventors show that TLR-4 expression is elevated in PBL from a subset of diabetic patients (FIG. 4). Accordingly, these observations suggest the possibility that by acting through positive paracrine and/or endocrine feedback signaling loop(s), inflammatory cells self-sensitize against an inflammatory stimuli.

In addition to TLR-4 mediated responses, a second inflammatory signaling system, known as the inflammasome, also plays a central role in diabetes. The inflammasome is a protein complex that includes caspase-1. Following recruitment to the complex, caspase-1 is auto-cleaved/activated. It is believed that activated caspase-1 then contributes to IL-1b and IL-18 processing and release. Endotoxin/TLR-4 signaling, in and of itself does not trigger caspase-1 activation. However, multiple stimuli, including, glucose and free fatty acids, reactive oxygen species, and mitochondrial DNA, can work in concert with TLR-4 ligands to induce caspase-1 activation. As shown in FIG. 4, the present inventors display the caspase-1 degradation in PBL from a subset of diabetes and high-risk prediabetic patients.

In another aspect of the present invention, the present inventors describe a robust increase in MMP9 expression in PBL from a subset of subjects at high risk for developing diabetes and diabetes patients, but not in PBL from healthy control subjects or non-diabetic patients (FIG. 4). Elevated plasma MMP9 and MMP2 levels were detected in diabetic patients. Furthermore, activated AMPK suppressed MMP9 expression in mouse embryonic fibroblasts. The relationship between MMP9 expression and AMPKα degradation in human PBL is currently undetermined.

In another aspect of the invention, the inventors introduce the use of protein expression profile of PBL as a powerful novel tool for tracking early-stage and asymptomatic prediabetic patients. In at least one embodiment, this tool might be sufficient in and of itself for diagnosis, or enhance the diagnostic power of small changes in fasting glucose levels or HbAlC. Furthermore, better understanding of mechanisms leading to the onset of MDS will provide an opportunity for the identification of new targets for therapy.

Metformin is currently the most frequently used drug for treatment of patients with diabetes. Building on the in vitro assay described in FIG. 3A, human blood were treated in vitro with metformin for 2 hours prior to the addition of endotoxin. FIG. 3C indicates that treatment with metformin alone induced AMPKα phosphorylation in human leukocytes. When combined with endotoxin, metformin prevented the increase in HIF-1α expression and AMPK α degradation, and instead induced AMPK α phosphorylation. These data demonstrate that the assay described in the present invention can be used to detect drug-induced changes relevant to diseases such as diabetes.

In another embodiment, the present invention provides biomarkers for the detection and tracking of chronic preclinical inflammatory responses. Advantageously, this enables or improves diagnosis of the disease even before the appearance of defined symptoms, shortening time to intervention, either clinically or via changes in lifestyle. Additionally, this enables a more objective and rapid assessment of treatment efficacy and strategy. These benefits will improve outcomes and significantly reduce patient care cost.

It is contemplated that chronic low-grade inflammatory responses trigger increases in HIF-1α, Glut3, autophagy, TLR-4, and MMP9 expression, and a parallel decline in AMPKα expression (due to AMPKα degradation) as well as caspase-1 activation/cleavage. In at least one aspect of the present invention, an effective treatment is shown to prevent, alter, and/or reverse these outcomes (see Table 1). Accordingly, in at least one embodiment, inventors describe methods of using effective inhibitors of low-grade inflammation to suppress HIF-1α, Glut3, and/or MMP9 expression, and prevent autophagy as well as AMPK degradation. In at least another embodiment the inventors describe in vitro methods of using effective inhibitors of low-grade inflammation to suppress HIF-1α, Glut3, and/or MMP9 expression, prevent autophagy and AMPK degradation in endotoxin-treated whole blood.

In a preferred embodiment, the assay of the present invention will focus on the detection of the N-terminal region of AMPKα (amino acids 1-251). This region will not be available for detection in blood samples treated with endotoxin or other inflammatory stimuli that trigger AMPKα degradation. Lack of detection will be indicative of disease, in at least one embodiment, samples can be screened simultaneously or in parallel for HIF-1α, Glut3, autophagy, MMP9, TLR4, caspase-1 and AMPKα expression, as well as AMPKα and caspase-1 cleavage.

TABLE 1

| | Control | Chronic | Expected outcome |
|---|---|---|---|
| | untreated | Inflammation- | if the test |
| | samples | like pattern | inhibitor is active |
| HIF-1α | Not expressed | Expressed | Not expressed |
| AMPKα | Is intact | Is degraded | Is intact |
| AMPK | No | No | Yes |
| phosphorylation | | | |
| Glut3 | Not expressed | Expressed | Not expressed |
| Autophagy | No | Yes | No |
| (increased | | | |
| LC3II:LC3I | | | |
| expression ratio) | | | |
| TLR4 | Expressed | Enhanced | Expressed |
| | | expression | |
| MMP9 | Not expressed | Expressed | Not expressed |
| Caspase 1 | intact | cleaved | intact |

In one embodiment, the assay proposed will focus on the detection of the N-terminal region of AMPKα (amino acids 1-251). This region will not be available for detection in PBL from subjects with chronic low-grade inflammation. Lack of detection will be indicative of a disease state. In this embodiment, samples can be screened simultaneously or in parallel for HIF-1α and/or Glut3 expression. In a preferred embodiment, diabetes will be manifested through lack of N-terminal AMPKα detection as well as detection of HIF-1α, Glut3 and/or MMP9. In yet another embodiment, the proposed assay will focus on the detection of MMP9. In another embodiment, the proposed assay is modified to focus on the detection of cleaved caspase-1.

The following non-limiting examples serves to further illustrate the present invention.

### EXAMPLES

### Example 1

### Expression of Novel Biomarkers of Low-Grade in Animation in Human Leukocytes

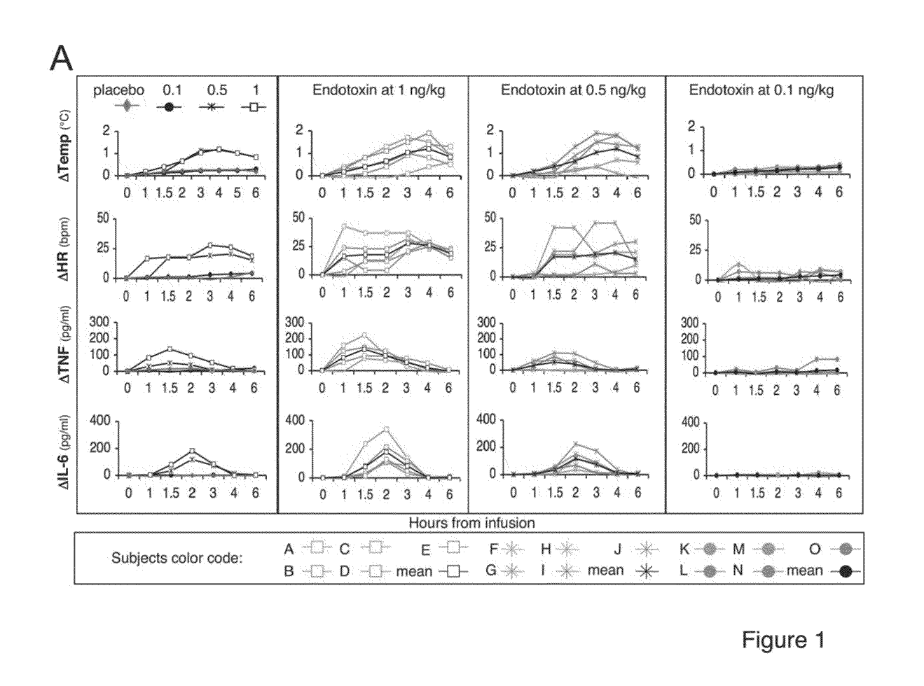

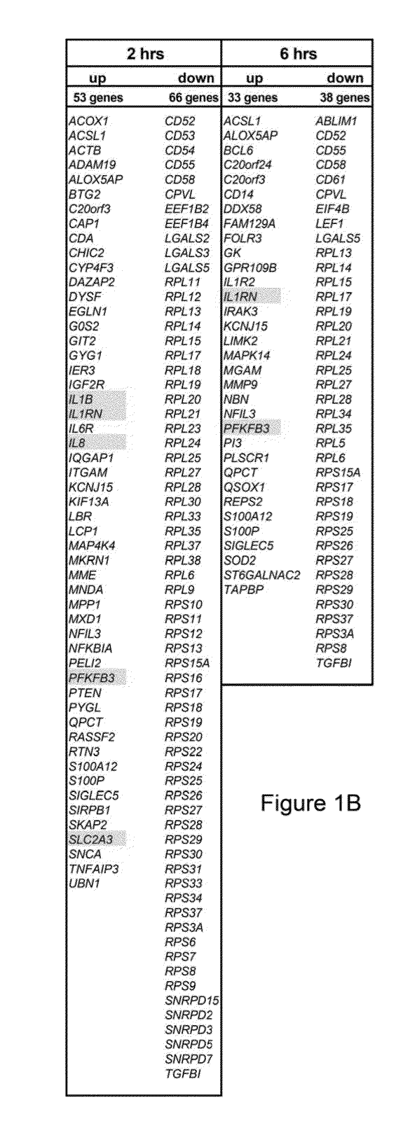

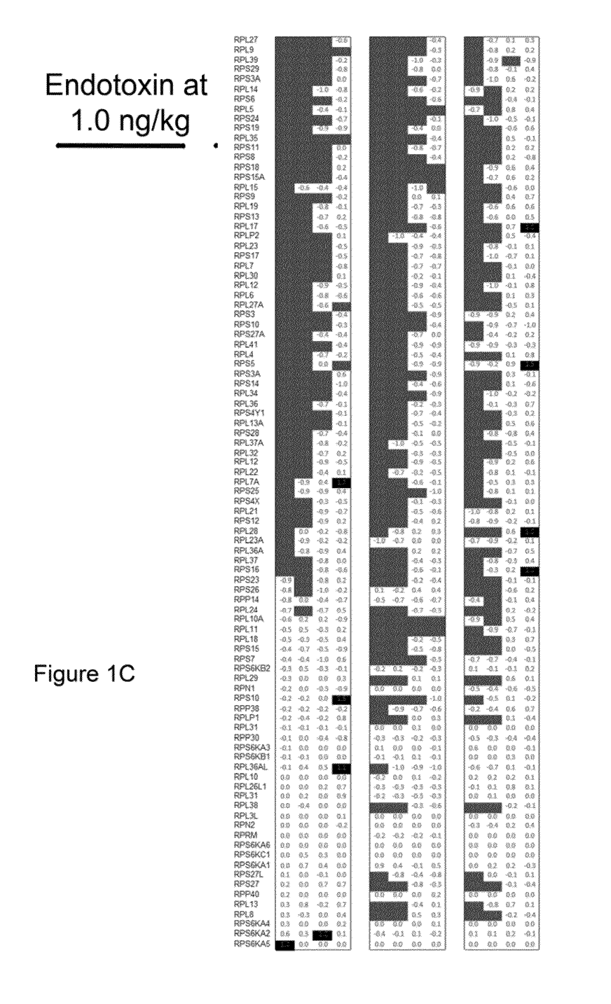

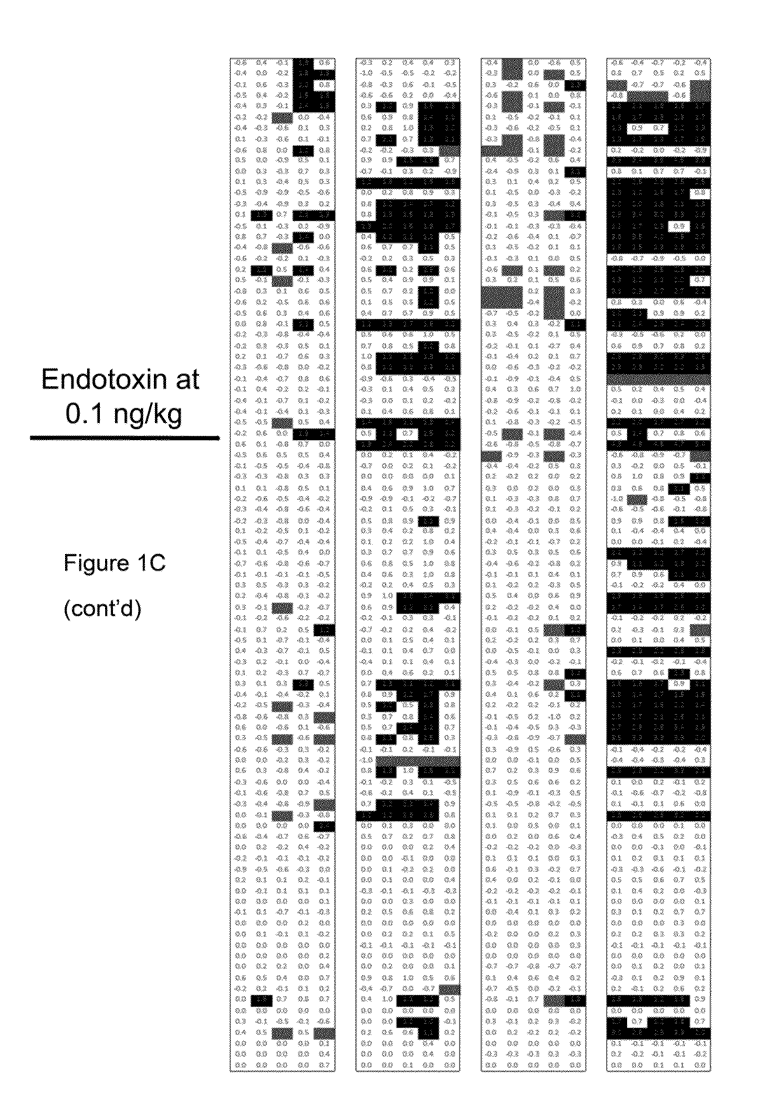

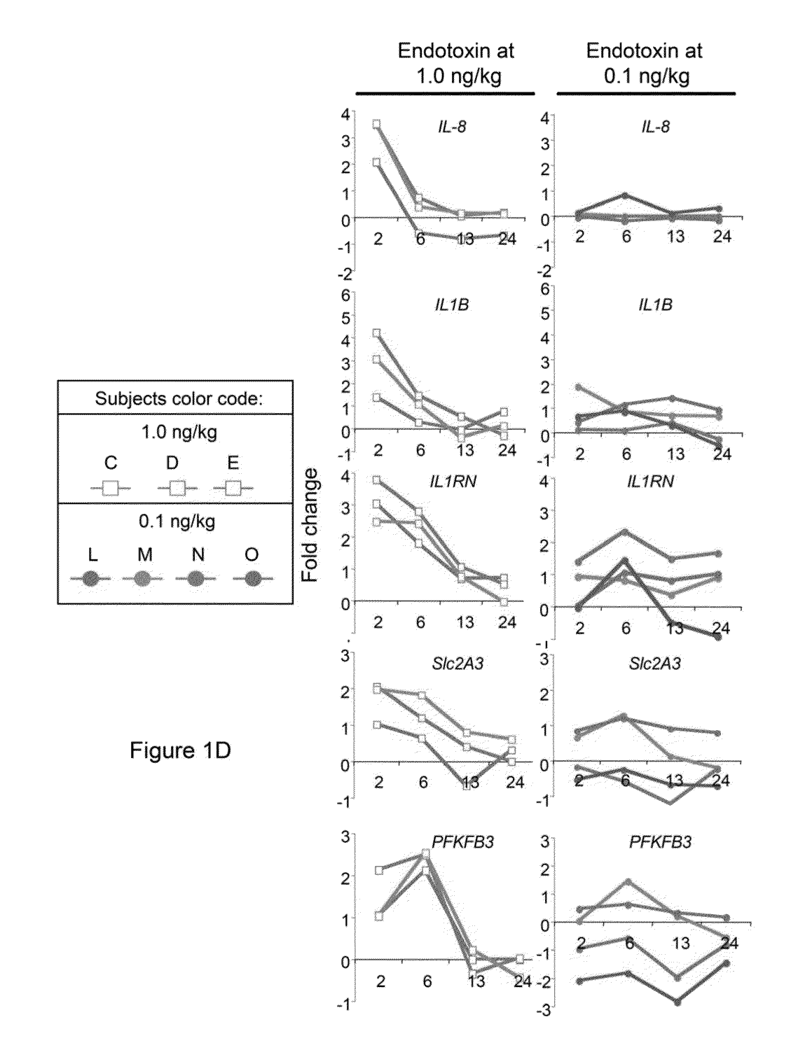

To characterize the endotoxemia response threshold in human subjects, volunteers were challenged with saline (control) or a bolus endotoxin dose of 0.1-, 0.5- or 1-ng/kg. Each subject (n=5 per group) was assigned a specific color/symbol code shown in FIGS. 1 and 2. Though variable in magnitude, all subjects administered endotoxin at 1 ng/kg exhibited characteristic increases in core temperature, heart rate (HR), and cytokines (FIG. 1A). Subjected challenged with endotoxin at 0.5 ng/kg exhibited attenuated responses, while those challenged with endotoxin at 0.1 ng/kg exhibited, on average, no response (FIG. 1A).

Prior genome-wide expression analyses identified numerous transcripts that were either induced or suppressed in leukocytes from human subjects challenged with endotoxin at 2- or 4-ng/kg Time-dependent expression analyses from the Focus Gene-Chip® microarrays (Affymetrix) representing 8,793 unique genes, identified approximately 175 genes that were either induced or suppressed in three subjects challenged with endotoxin at 1 ng/kg (FIG. 1B). The vast majority of these common transcripts represented BPL and RPS genes encoding proteins associated with the large (RPL) or small (RPS) ribosomal subunits (10) (FIGS. 1B and 1C). By 2 hours post challenge, 56 of the total 94 RPL/RPS genes present on the Focus Gene-Chip® microarray were suppressed in these subjects (FIGS. 1B and 1C). The commonly induced transcripts include several that are associated with cytokine production (IL8, IL1B, IL1RN), and two (Slc2A3 and PFKFB) that are associated with glycolysis (FIGS. 1C and 1D). Slc2A3 and PFKFB3 encode the glucose transporter Glut3 and 6-phosphofructo-2-kinase, respectively. Remarkably, no transcript was either induced or suppressed in all four subjects challenged with endotoxin at 0.1 ng/kg. Collectively, these data demonstrate that an in vivo endotoxin dose greater than 0.1 ng/kg is required to initiate a consistent systemic, transcriptional, or cytokine response.

Endotoxin Effects in Human Leukocyte

In this aspect of the invention, the inventors studied the in vivo bioenergetics response to endotoxin at doses of 1.0 and 0.1 ng/kg, to determine whether low-dose endotoxemia can induce metabolic dysfunction. Administration of in vivo endotoxin at a dose of 2 ng/kg triggers profound metabolic perturbations in human leukocytes, including a decrease in ATP concentration and an increase in autophagy. The inventors have previously observed that when administered to mice, in vivo endotoxin induced within minutes (˜10 minutes) a decline in both AMP-activated protein kinase α subunit (AMPKα) and Sirt1 expression in leukocytes and liver. This was followed within 90 minutes by parallel temporal increases in HIF-1α expression and autophagy, and a decline in ATP levels.

After isolation of whole blood leukocytes from a subset of the subjects described in FIG. 1, temporal changes in ATP levels, autophagy, as well as changes in AMPKα, HIF-1α and Glut3 protein expression were examined both pre-(0 hr) and post-endotoxin bolus, at the time points indicated. The leukocytes obtained from those subjects post-endotoxin challenge (1 ng/kg) exhibited a decline in ATP levels reaching a nadir between 2-6 hours post-infusion (FIG. 2). This was accompanied by changes in LC3-1/LC3-II expression, indicative of an increase in autophagy (FIG. 2). In parallel, HIF-1α and Glut3 expression increased over the same time frame, supporting our previous observations in mice.

A decline in ATP levels is expected to activate AMPK, a key cellular energy sensor. The AMPK activation requires AMPKα phosphorylation at threonine residue 172. Contrary to what was expected, the inventors found that leukocyte AMPKα expression declined quickly and abruptly (FIG. 2). Further, the ˜63 kD AMPKα protein band was replaced by two smaller protein bands of approximately 55-kD and 35-kD, demonstrating that AMPKα is rapidly and transiently degraded in human leukocytes following an in vivo endotoxin challenge.

Next, the inventors analyzed whole blood leukocytes obtained from the subjects cohort challenged with 0.1 ng/kg endotoxin (FIG. 2B). Unexpectedly, bioenergetics and protein-expression changes (FIG. 2B), that were qualitatively indistinguishable from those induced by endotoxin at 1 ng/kg were identified. For the first time, the present inventors confirm that a TLR-4 ligand can trigger profound changes in cellular big in the absence of systemic inflammatory indicators.

Ex Vivo Endotoxin Induced Response in Human Leukocytes

To determine whether the in vivo effects of endotoxin could be reproduced in an ex vivo system, leukocytes isolated from whole blood stimulated with endotoxin ex vivo (10 ng/ml) exhibited metabolic perturbations that were identical to those observed in leukocytes obtained from subjects who had been challenged with endotoxin in vivo (FIG. 3). Furthermore, as documented m endotoxin-challenged mice liver, AMPKα expression declined within minutes (˜10 min) after the addition of endotoxin (FIG. 3A). This was followed by the appearance of HIF-1α and Glut3 expression by 90 minutes post-challenge, increased autophagy and a decline in ATP levels. Remarkably, the endotoxin-induced bioenergetics dysfunction detected in leukocytes lasted for approximately 6 hours irrespective of whether the endotoxin challenge occurred in vivo or ex vivo. These data establish that endotoxin challenge induces a single monophasic bioenergetics response of a set duration in human leukocytes.

To examine the signaling events linking endotoxin induced responses ex vivo with the altered metabolic/bioenergetic profile, the inventors examined the effect of pharmacological inhibitors implicated in the regulation of HIF-1α expression in other model systems. As shown in FIG. 3B, the PI-3K inhibitor LY294002, inhibited the endotoxin-induced changes in AMPKα and HIF-1α expression, as well as autophagy and the decline in ATP levels. In contrast, the mTOR inhibitor, rapamycin, and two types of HIF-1 inhibitors, acriflavine (ACF) and YC-1 (3-(5′-hydroxymethyl-2′-furyl)-1-benzylindazole) inhibited autophagy and the decline in ATP levels, but did not prevent AMPKα degradation. These data identify PI3-kinase as the common upstream regulator of endotoxin-induced bioenergetics dysfunction in leukocytes (FIG. 3B). Furthermore, they indicate that distinct PI-3K effectors regulate HIF-1α stabilization and AMPKα degradation.

To examine the effect of metformin on human leukocytes bioenergetics, whole blood samples were pretreated with metformin ex vivo prior to the addition of endotoxin. Metformin not only induced AMPKα phosphorylation at Thr 172 (FIG. 3C), but also inhibited endotoxin-induced AMPKα degradation as well as subsequent HIF-1α expression. These data are the first to show that metformin can protect leukocytes from metabolic perturbations induced by low-level endotoxin. Recent studies revealed that diabetics treated with metformin have from 25-40% less cancer than those treated with insulin. Since HIF-1α expression is central to cancer cell survival, the inventors for the first time effectively show that the beneficial effects of metformin in cancer prevention are associated, at least in part, to its ability to inhibit HIF-1α expression.

In another aspect of the present invention, inventors show that while LY294002 and metformin prevented AMPKα degradation, SRT1720 did not (FIG. 3D). Sirt1 activators that include resveratrol and SRT1720 have anti-inflammatory activities. Resveratrol pretreatment prevents Sirt1 degradation, HIF-1α expression, and autophagy, but not AMPKα degradation, in the liver following endotoxin challenge in a murine model.

In contrast, all three pharmacologic agents prevent autophagy, HIF-1α and IkBα degradation. These data indicate that AMPKα is not the primary target of SRTI720 in endotoxin-challenged cells and tissues, but rather that SRTI720 can block HIF-1α expression independent of AMPKα. Collectively, these data establish that endotoxin-mediated signaling events induce AMPKα degradation followed by stabilization of HIF-1α expression in human leukocytes. Furthermore, the data indicate that ex vivo, metformin prevents both outcomes.

Even though both HIF-1 and AMPK were implicated in the regulation of glycolysis, autophagy, glucose transporters, and even PFKFB3 expression, the early and rapid decline in AMPKα expression, and the subsequent increase in HIF-1α expression, reported here, provides that HIF-1α is the central regulator of cellular bioenergetics downstream of TLR-4.

The Link Between Low-Endotoxin Levels and Diabetes

To establish a link between human responses to low-endotoxin levels and diabetes, LC3-I/LC3-II (autophagy), HIF1α, and AMPKα expression were compared in leukocytes from diabetic patients, as defined by a HbAlC>6.4 or under treatment for diabetes, and non-diabetic patients. Blood, samples were obtained using a double-blinded approach. The patients profile is described in Table 2. Increases in LC3-II, HIF-1a, TLR4 and MMP9 expression, as well as AMPKa cleavage and caspase-1 cleavage/activation were all detected in 7 of 13 Type 2 diabetes patients and 3 of 3 Type 1 diabetes patients.

The present data establish a new TLR-4-ligand induced response characterized by cellular bioenergetics dysfunction in human leukocytes following sub-clinical endotoxin challenge in vivo, with hallmarks that include AMPKα degradation, increased HIF-1α expression and autophagy. More importantly, the sub-clinical endotoxin dose observed is below the threshold associated with the typical systemic inflammatory response.

Further, the inventors effectively show that these newly described hallmarks of leukocyte bioenergetics dysfunction are detectable in leukocytes from a subset of T2DM patients (FIG. 4). Declines in skeletal muscle mitochondrial content and function, as well as ATP levels, were documented in elderly T2DM diabetes patients. However, the mechanism by which the bioenergetics dysfunction is induced in patients with diabetes remains uncharacterized. The data shown by the inventors is therefore the first to suggest a mechanistic association between metabolic endotoxemia, AMPKα degradation, increased HIF-1α expression, and the bioenergetics impairment associated with T2DM.

### Example 2

### Changes in Expression of TLR-4 Signaling Components in Human Leukocytes.

Materials and Methods

Antibodies, reagents, and Inhibitors

The following antibodies were used at the indicated dilution: LC3 (L7543; 1:500) and actin (A2066; 1:1000) from Sigma. IRS-1 (sc-559: 1:1000), HIF-1α (sc-10790; 1:250), Akt (sc-5298; 1:500), and TLR4 (sc-10741; 1:500) from Santa Cruz Biotechnology, phospho-IRS-1 (Ser307) (#2381; 1:1000), phospho-Akt (Thr308) (#9275; 1:1000), phospho-Raptor (ser792) (#2083; 1:1000), Raptor (#2280; 1:200), and Phospho-p70 S6 Kinase (Thr389) (#9205; 1:1000) were from Cell signaling Technology.

The source of reagents and final concentrations used in this example are as follows:

Endotoxin (lipopolysaccharide from Escherichia coli 0111B4, Sigma, 10 ng/ml). Insulin (Humalog Mix 50/50; Lilly) was used at the indicated concentrations (0.05-1 unit/ml), Polymyxin (Invivogen, 50 μg/ml), CLI-095 (Invivogen; 3 μM), LY294002 (Cayman Chemical; 10 μM), Rapamycin (Tocris Bioscience; 100 nM), YC-1 (Sigma; 30 μM), Acriflavine (ACF; Sigma; 5 μM)

Human Subjects were enrolled in the study following the Institutional Review Board of Rutgers Robert Wood Johnson Medical School approval. Written informed consent was obtained from all participates prior to inclusion in the study. Blood was drawn into EDTA-containing tubes. To isolate leukocytes, lysis buffer (bicarbonate-buffered ammonium chloride solution, 0.826% NH₄Cl, 0.1% KHCO₃, 0.0037% Na₄EDTA in H₂O) was added at a ratio of 20:1 (lysis buffer/blood). Once the erythrocytes lysed, the samples were centrifuged for 10 mM at 400×g. The leukocyte pellet was washed once with phosphate-buffered saline. The pellet was suspended in RIPA buffer (1% Triton X-100, 1% deoxycholic acid, 10 mM Tris-HCl, pH 7.2, 158 mM NaCl, 0.1% SDS, and 1 mM PMSF and complete protease inhibitor mixture (Roche Applied Science)). Lysates containing equal protein amounts were analyzed by immunoblotting. Where indicated, endotoxin, insulin, and/or inhibitors were added to the whole blood samples prior to leukocyte isolation.

For the plasma mixing experiments, the blood samples were sedimented at unit gravity for 1.5 hours. The upper plasma fraction was recovered leaving the cellular fraction intact. The plasma fraction was next centrifuged at 1800×g to remove residual cells. The plasma and cellular fractions from the non-diabetic donors and the patients were mutually exchanged. Thus, plasma derived from the non-diabetic donor was added to diabetic patient cellular fraction and vis e versa. The samples were mixed gently end-over-end and incubated for the specified time. The leukocytes were subsequently isolated, lysed, and analyzed by immunoblotting.

Leukocyte Biomarker Expression

Leukocytes, which include neutrophils and monocytes, are key mediators of host-inflammatory responses. The possibility that circulating leukocytes from diabetic patients exhibit changes in expression level or phosphorylation state of proteins that are associated with the TLR4 signaling pathway has riot been explored. To address this possibility, leukocyte samples were obtained from diabetic patients as defined by a HbAlC>6.4 or under treatment for diabetes (n=22; 7 type 1 and 15 type 2), and non-diabetic patients (n=10) (clinical characteristics and demographics of the study participants are shown in Table 2) were analyzed.

TABLE 2
Clinical characteristics and demographics of non-diabetic
(N), type 1 diabetes (T1D), and type 2 diabetes (T2D) study
participants. Abbreviations used: NA, Not available: Met,
Metformin; Ins, Insulin; HbA1C, hemoglobin A1C

| | | | Fasting | | | |
|---|---|---|---|---|---|---|
| Subject | | Diabetes | plasma | | | Age |
| ID # | Diagnosis | Medication | glucose | HbA1C | Sex | (years) |
| Cntrl | Non-diabetic | | 88 | N.A. | F | 55 |
| 1 | Non-diabetic | | N.A. | N.A. | M | 24 |
| 2 | T2D | Met | 69 | 5.9 | M | 64 |
| 3 | Non-diabetic | | 78 | 5.7 | M | 77 |
| 4 | T2D | Ins | 63 | 6 | M | 62 |
| 5 | Non-diabetic | | 76 | N.A. | F | 75 |
| 6 | Non-diabetic | | 48 | 5.9 | F | 47 |
| 7 | T2D | Ins | 66 | 8.2 | M | 65 |
| 8 | T2D | Met | 64 | 8 | M | 64 |
| 9 | T2D | Met | 67 | 7.4 | M | 67 |
| 10 | T2D | Met + Ins | 49 | 9.2 | F | 49 |
| 11 | Non-diabetic | | 69 | N.A. | M | 69 |
| 12 | T2D | None | 51 | 6.7 | M | 51 |
| 13 | T2D | None | 69 | 6.6 | F | 69 |
| 14 | Non-diabetic | | 49 | 5.5 | F | 49 |
| 15 | Non-diabetic | | 65 | N.A. | F | 64 |
| 16 | T1D | Ins | 99 | 6.1 | M | 71 |
| 17 | Non-diabetic | | 117 | 6 | M | 52 |
| 18 | T2D | Ins | 294 | 8.7 | M | 63 |
| 19 | T2D | Met | 79 | 8.3 | M | 48 |
| 20 | T2D | Met + Ins | 149 | 10.4 | M | 56 |
| 21 | T1D | Ins | 71 | 6.2 | M | 41 |
| 22 | Non-diabetic | | 116 | 6.3 | M | 62 |
| 23 | T2D | Ins | 176 | 8.8 | M | 64 |
| 24 | T2D | M | 152 | 7.2 | M | 69 |
| 25 | T1D | Met + Ins | 269 | 11.3 | M | 25 |
| 26 | T2D | M | 166 | 9.3 | M | 57 |
| 27 | T1D | Ins | N.A. | 7.2 | M | 28 |
| 28 | T1D | Ins | 112 | 8.3 | F | 57 |
| 29 | T2D | Ins | N.A. | N.A. | M | 38 |
| 30 | T1D | Ins | 211 | 9.7 | M | 62 |
| 31 | T1D | Ins | 79 | 6.5 | M | 26 |

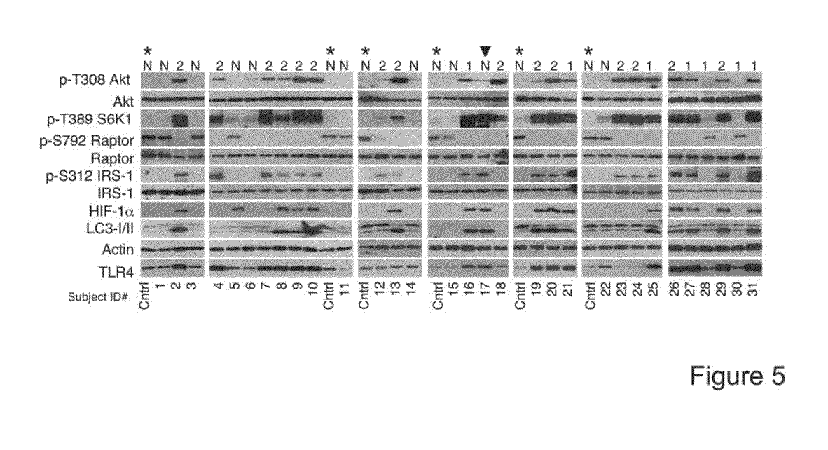

Among the participating patients, 71% of type 1 and 100% of type 2 diabetics, showed Akt phosphorylated at Thr-308 (pAkt), S6K1 phosphorylated at Thr-389 (pS6K1), and Raptor dephosphotylated at Ser-792, which is a site important for mTOR inhibition (FIG. 5). The increase in pS6K1 correlated with an increase in IRS-1 phosphorylation at Ser-312 (Ser-307 in mice) (pIRS-1), a site associated in the regulation of insulin signaling, in all but one type 2 diabetic patients. The HIF-1α levels and enhanced autophagy, indicated by the increase in LC3-II expression, were detected in 71% of type 1 and 66% of type 2 diabetic patients' samples. In marked contrast, 9 of the 10non-diabetic individuals were without any indication of activation of these key signaling intermediates. The one patient initially classified, as non-diabetic patient (patient ID #17), who exhibited pAkt, pS6K1, Raptor dephosphorylation, pIRS-1, HIF-1α and autophagy turned out to have a HbAlC of 6% and fasting, glucose of 117 mg/dL.

These data establish that leukocytes from a majority of type 1 and type 2 diabetic patients but not non-diabetic individuals demonstrate a characteristic pattern of activation of multiple indicators of growth factor and cytokine signaling.

Plasma from Diabetic Patients Contains a Soluble Component that Can Activate Leukocytes from Non-Diabetic Patients

To determine whether the signaling phenotype of type 1 and type 2 diabetic patient leukocytes was cell-intrinsic, or reflected the presence of a circulating factor, a plasma mixing experiment was undertaken. Blood samples from a non-diabetic control and a diabetic patient that exhibited the markers described in FIG. 5, including pS6K1, pIRS-1, and HIF-1α, were separated into plasma and cellular fraction and then mutually exchanged; thus, plasma derived from the non-diabetic donor blood was added to diabetic patient cellular fraction, and vise versa.

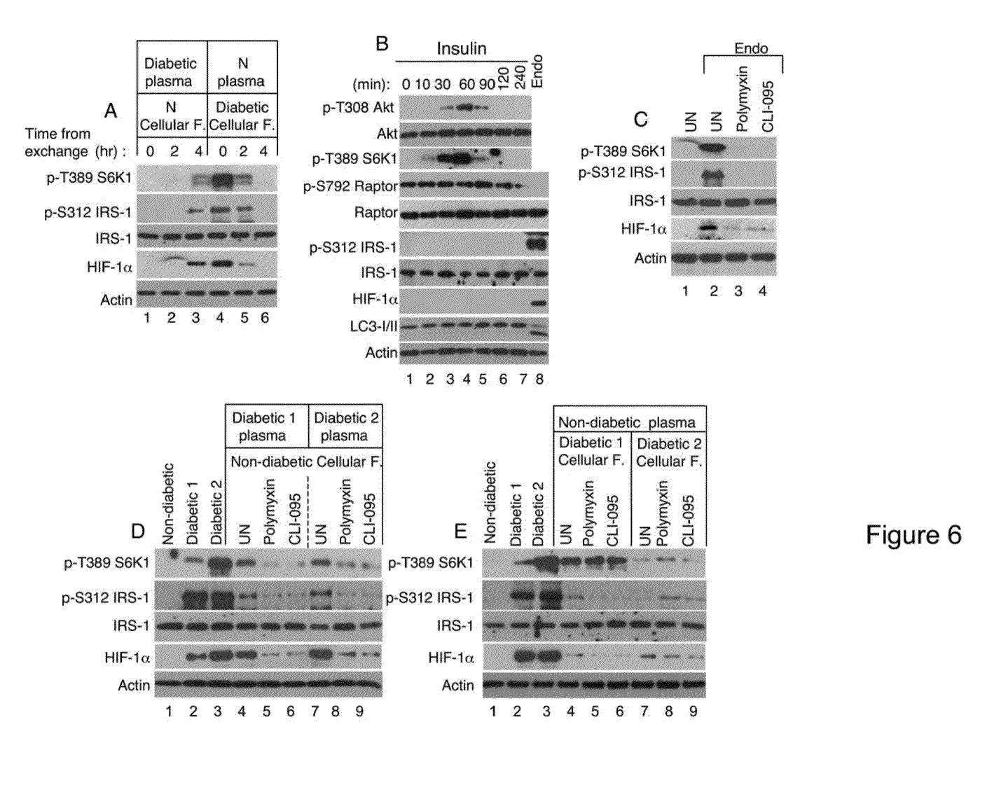

Leukocytes were then isolated and analyzed at two time points post-exchange (FIG. 6). By four hours post-exchange, the non-diabetic donor leukocytes exhibited pS6K1, pIRS-1, and HIT-1α expression, as initially seen in the diabetic patient leukocytes (FIG. 6A). In marked contrast, pS6K1 pIRS-1, and HIF-1 a expression progressively declined in the diabetic patient leukocytes during incubation with the non-diabetic donor plasma. These findings establish the presence of a soluble and transmissible leukocyte activator within diabetic patient plasma, and that up-regulation of pS6K1, pIRS-1, and HIF-1α expression in diabetic patient leukocytes requires the persistent presence of this plasma component.

Insulin Does Not Trigger the Expression of the Complete Pattern of Biomarkers Detected in Leukocytes from Diabetic Patients

To establish the nature of the circulating factor enriched, in plasma from diabetics, multiple candidates were considered. As leukocytes express insulin receptor and circulating insulin is high in many type 2 and treated type 1 diabetic patients, insulin was chosen as the primary candidate. However, the insulin-signaling pathway in leukocytes is poorly characterized. In whole blood from non-diabetic individuals, insulin triggered increases in pAkt and pS6K1, which peaked between 30 and 90 min post-treatment (FIG. 6B). However, insulin failed to induce Raptor dephosphorylation, pIRS-1, or increases in HIF-1α expression and autophagy. The inability of insulin to reproduce the complete pattern of altered signaling provoked in leukocytes from diabetic patients reveals that it is unlikely that insulin alone is the responsible circulating factor.

Evidence of Endotoxin or an Endotoxin-Like Components in Diabetic Patients Plasma

To determine whether endotoxin is the component in diabetic patient plasma that regulates leukocyte activation, endotoxin/TLR4 signaling was blocked using two pharmacologic inhibitors, polymyxin and. CLI-095. Polymyxin is a natural polypeptide antibiotic that binds the lipid A moiety of endotoxin, and consequently interferes with endotoxin binding to TLR4. CLI-095, also known as TAK-242, is a specific TLR4 signaling inhibitor. As leukocytes express multiple Toll-like receptors (TLR) including TLR4, which binds endotoxin, TLR4 functions in the regulation of Akt/S6K1/mTOR axis is of interest.

Pretreatment of non-diabetic donor blood with polymyxin or CLI-095 prevented pS6K1, pIRS-1, and HIF-1α expression upon challenge with endotoxin in vitro (FIG. 6C) demonstrating that these outcomes are TLR4-dependent. Next, non-diabetic donor leukocytes and leukocytes from two type 2 diabetic patients were either untreated, or treated with either polymyxin or CLI-095 prior to the plasma/leukocytes exchange. As shown above, pS6K1, pIRS-1, and HIF-1α expression were all detected in non-diabetic donor leukocytes following exposure to diabetic patients plasma. Both polymyxin and CLI-095 inhibited these responses (FIG. 6D). The non-diabetic patient plasma did not trigger increases in pS6K1, pIRS-1 or HIF-1α expression following incubation with the diabetic patients leukocytes (FIG. 6E). These data establish endotoxin or an endotoxin-like molecule as a leukocyte activation component present in diabetic patient plasma.

The Endotoxin-Induced Signaling Pathway in Leukocytes

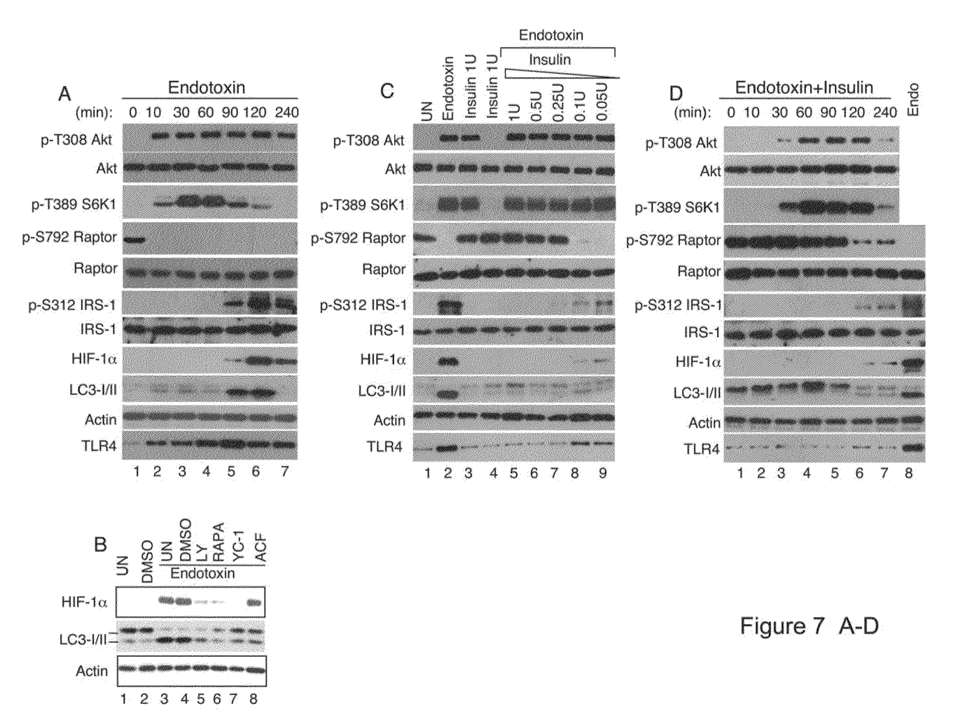

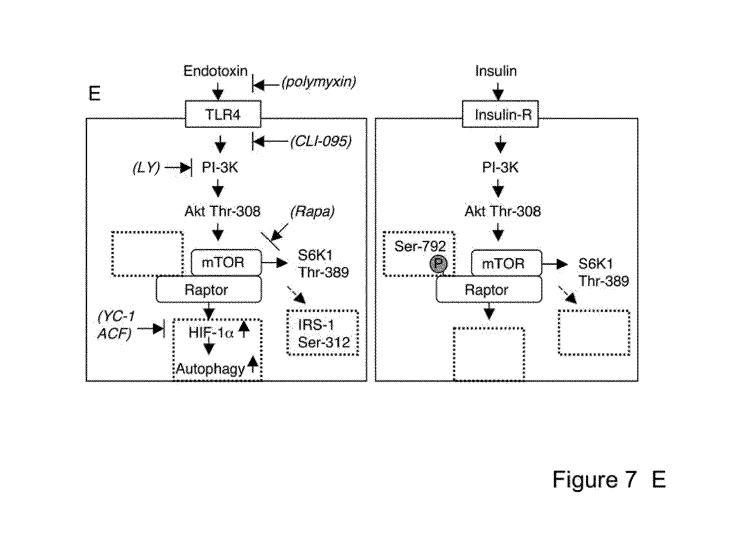

Endotoxin's capability to trigger protein expression changes similar to those that are observed in type 1 and type 2 diabetic patients was studied. Accordingly, treatment of human leukocytes in vitro with endotoxin for 10 minutes led to increased pAkt, pS6K1, and Raptor dephosphorylation (FIG. 7A). Phosphorylation of S6K1 was then followed by a rapid increase in IRS-1 on serine residue 312. The PI-3K inhibitor, LY294002, and the mTORC1 inhibitor, rapamycin, blocked HIF-1α expression and autophagy in endotoxin-treated leukocytes (FIG. 7B). Similarly, the HIF-1α activation inhibitor, YC-1, and HIF-1 dimerization inhibitor, acriflavine (ACF), prevented autophagy after challenge.

These data demonstrate a TLR4-dependent signaling pathway beginning with Akt and mTOR activation and culminating with the appearance of HIF-1α and autophagy in leukocytes over the course of four hours (FIG. 7B). In addition, endotoxin was found to increase TLR4 expression in leukocytes, indicating a positive feedback loop (FIG. 7A). This observation was further reflected in vivo by elevated TLR4 expression in 71% of type 1 and 73% of type 2 diabetic patient blood samples (FIG. 5). Increased TLR4 expression has also been noted in muscle from insulin-resistant subjects. Collectively, the data presented herein establish that the entire repertoire of activated signaling components identified in type 1 and type 2 diabetic patient leukocytes can be accounted for by endotoxin engagement of TLR4.

Insulin Antagonizes the Endotoxin-Induced Responses in Human Leukocytes

Following food intake, especially if high in fat, human leukocytes might be exposed to increases in both insulin and endotoxin. Therefore, the combined effects of insulin and endotoxin on leukocytes were examined using the in vitro whole blood system. Dose-dependent studies revealed that insulin at a dose higher than 0.1 unit/ml suppressed the dephosphorylation of Raptor, pIRS-1, HIF-1α expression, autophagy, as well as TLR4 expression in endotoxin-treated leukocytes (FIG. 7C). Insulin at a dose as low as 0.1 unit/ml also delayed onset of pAkt and pS6K1 in endotoxin-treated leukocytes (FIG. 7D). As HIF-1α regulates the production of proinflammatory cytokines in endotoxin challenged mice, the robust inhibitory effect of insulin on HIF-1α expression suggests the possibility that insulin has anti-inflammatory effects and antagonizes a subset of endotoxin-induced inflammatory responses in human subjects challenged parenterally with endotoxin.

These data establish that insulin is a negative regulator of TLR4-mediated responses in human leukocytes, raising the possibility that insulin may protect leukocytes and organs from low-grade endotoxin-induced inflammatory responses. The present data also establish the presence of a signaling signature in diabetic patient leukocytes that is indicative of endotoxin- and TLR4-dependent metabolic reprogramming. Since a low endotoxin dose (0.1 ng/kg) delivered parenterally to humans failed to elicit systemic inflammatory responses but triggered profound metabolic changes in peripheral blood leukocytes, including a decline in ATP levels, HIF-1α expression and autophagy, the present data suggests that insulin may play a central role in suppressing low-grade endotoxin-induced responses.

One of ordinary skill in the art can appreciate the possibility that chronic endotoxin levels initiate a feed-forward TLR4-dependent signaling cascade that increases leukocyte susceptibility to endotoxin triggering an inflammatory state that insulin can no longer overcome. In summary, the present invention highlights the concept that circulating leukocytes provide a powerful novel tool for defining etiologic determinants of inflammatory related conditions such as diabetes in humans. Present inventors are first to show this tool and use thereof for diagnosis of diabetic patients, diabetic stage classification of patients, monitoring the disease progression, and further identifying new therapeutic targets.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. A number of embodiments of the invention have been described. Nevertheless, it will he understood that various modifications may be made without departing from the spirit and scope of the invention. Accordingly, other embodiments are within the scope of the following claims.

1. A method for diagnosing an inflammatory condition in a subject, the method comprising assaying leukocytes from the subject for a biomarker selected from the group consisting of AMPKα, HIF-1α, Glut3, TLR4, caspase 1, MMP9 and any combinations thereof.

2. The method according to claim 1, wherein said biomarker is HIF-1α.

3. The method according to claim 1, wherein said biomarker is Glut3.

4. The method according to claim 1, further comprising assaying said leukocytes for autophagy.

5. The method according to claim 1, wherein said biomarker is TLR4.

6. The method according to claim 1, wherein said biomarker is caspase 1.

7. The method according to claim 1, wherein said biomarker is MMP9.

8. (canceled)

9. The method according to claim 1, wherein the assaying of said leukocytes determines the change in expression of said biomarkers.

10. The method according to claim 1, wherein the inflammatory condition is diabetes.

11. A method for screening an agent to determine its ability to modulate an inflammatory response in a subject, the method comprising contacting at least one leukocyte from the subject with the agent and assaying the at least one leukocyte for expression of a protein selected from the group consisting of AMPKα HIF-1α, Glut3, TLR4, caspase 1, MMP9 and any combinations thereof.

12. The method according to claim 11, wherein said protein is HIF-1α.

13. The method according to claim 11, wherein said protein is Glut3.

14. (canceled)

15. The method according to claim 11, wherein said protein is TLR4.

16. The method according to claim 11, wherein said protein is caspase-1.

17. The method according to claim 11, wherein said protein is MMP9.

18. The method according to claim 11, wherein the inflammatory response is diabetes.

19. The method of claim 1, wherein the expression level of at least one leukocyte biomarker are measured by the assay.

20. The method of claim 19, wherein the measured expression level indicates elevation of ATP, degradation of AMPKα, expression of HIF-1α, activation of caspase-1, enhanced expression of TLR4 or MMP9, or any combinations thereof.

21. The method of claim 9, wherein the change in expression of said biomarkers indicates elevation of ATP, degradation of AMPKα, expression of HIF-1α, activation of caspase-1, enhanced expression of TLR4 or MMP9, or any combinations thereof.
